(19) United States
(12) Patent Application Publication
(10) Pub. No.: US 2024/0371488 A1
(43) Pub. Date: Nov. 7, 2024

(54) MEDICINE RECOMMENDATION APPARATUS, CONTROL METHOD, AND COMPUTER READABLE MEDIUM

(71) Applicants: NEC CORPORATION, Tokyo (JP); OSAKA UNIVERSITY, Suita-shi, Osaka (JP)

(72) Inventors: Masataka KIKUCHI, Osaka (JP); Yasushi MATSUMURA, Osaka (JP); Kenichi KAMIJO, Tokyo (JP); Yasuto FUSHIMI, Tokyo (JP); Kaori KOBAYASHI, Tokyo (JP)

(73) Assignees: NEC CORPORATION, Tokyo (JP); OSAKA UNIVERSITY, Suita-shi, Osaka (JP)

(21) Appl. No.: 18/288,746

(22) PCT Filed: Apr. 28, 2021
(86) PCT No.: PCT/JP2021/016879

§ 371 Date: Oct. 27, 2023

(51) Int. Cl. G16H 20/10; G16H 70/40

(52) U.S. Cl. CPC G16H 20/10; G16H 70/40

(57) ABSTRACT

A medicine recommendation apparatus (2000) acquires disease information (20), gene mutation information (30), and current medication information (40). The disease information (20) is information regarding a disease that a subject suffers from. The gene mutation information (30) is information regarding a gene mutation of the subject. The current medication information (40) is information regarding a medicine that the subject is using. The medicine recommendation apparatus (2000) generates candidate medicine information (10) by using the disease information (20), the gene mutation information (30), and the current medication information (40). The candidate medicine information (10) is information regarding a candidate for a medicine use of which can be recommended to the subject or which can be prescribed to the subject.

### TECHNICAL FIELD

The present disclosure relates to a technology for assisting a doctor, a pharmacist, or the like in selecting a medicine.

### BACKGROUND ART

Medicines suitable for individual persons or animals are prescribed or recommended (hereinafter, expressed as “prescribed or the like”) to them. Further, systems for assisting prescription or the like of medicines have been developed. For example, Patent Literature 1 discloses a system capable of, when a medicine is prescribed, taking a side effect of the medicine into consideration.

### CITATION LIST

### Patent Literature

Patent literature 1: Japanese Unexamined Patent Application Publication No. 2002-245172

### SUMMARY OF INVENTION

### Technical Problem

What needs to be considered when a medicine is prescribed is not limited to side effects. The present invention has been made in view of the above-described problem, and an objective thereof is to provide a technology for enabling a medicine to be prescribed or recommended more appropriately.

### Solution to Problem

A medicine recommendation apparatus according to the present disclosure includes: an acquisition unit configured to acquire disease information that is regarding a disease that a subject suffers from, gene mutation information that is regarding a gene mutation of the subject, and current medication information that is regarding a medicine that the subject is using; and a generation unit configured to generate candidate medicine information by using the disease information, the gene mutation information, and the current medication information, the candidate medicine information being information regarding a candidate for a medicine to be recommended to the subject to use or to be prescribed to the subject.

A control method according to the present disclosure is performed by a computer. The control method includes: an acquisition step of acquiring disease information that is regarding a disease that a subject suffers from, gene mutation information that is regarding a gene mutation of the subject, and current medication information that is regarding a medicine that the subject is using; and a generation step of generating candidate medicine information by using the disease information, the gene mutation information, and the current medication information, the candidate medicine information being information regarding a candidate for a medicine to be recommended to the subject to use or to be prescribed to the subject.

A computer readable medium according to the present disclosure stores a program for causing computer to perform a control method according to the present disclosure.

### Advantageous Effects of Invention

According to the present disclosure, a technology for enabling a medicine to be prescribed or recommended more appropriately is provided.

### BRIEF DESCRIPTION OF DRAWINGS

FIG. 1 shows an example of an overview of operations performed by a medicine recommendation apparatus according to a first example embodiment;

FIG. 2 is a block diagram showing an example of a functional configuration of a medicine recommendation apparatus according to the first example embodiment;

FIG. 3 is a block diagram showing an example of a hardware configuration of a computer that implements a medicine recommendation apparatus;

FIG. 4 is a flowchart showing an example of a flow of processes performed by the medicine recommendation apparatus according to the first example embodiment;

FIG. 5 shows an example of first knowledge information in the form of a table;

FIG. 6 shows an example of first knowledge information further containing information about attributes;

FIG. 7 shows an example of second knowledge information in the form of a table;

FIG. 8 shows an example of second knowledge information further containing information about attributes;

FIG. 9 shows an example of third knowledge information in the form of a table;

FIG. 10 shows an example of third knowledge information further containing information about attributes;

FIG. 11 conceptually shows a process for generating candidate medicine information;

FIG. 12 shows an example of a case in which candidate medicine information indicates ranks;

FIG. 13 is a first diagram showing an example of candidate medicine information including information about resistances and side effects; and

FIG. 14 is a second diagram showing an example of candidate medicine information including information about resistances and side effects.

### EXAMPLE EMBODIMENT

An example embodiment according to the present disclosure will be described hereinafter in detail with reference to the drawings. Components corresponding to or the same as each other are assigned the same or corresponding numerical numbers (or symbols) throughout the drawings, and redundant descriptions thereof are omitted as appropriate. Further, unless otherwise described, pre-defined values such as predetermined values and thresholds are stored in advance in a storage device or the like accessible from an apparatus that uses these values.

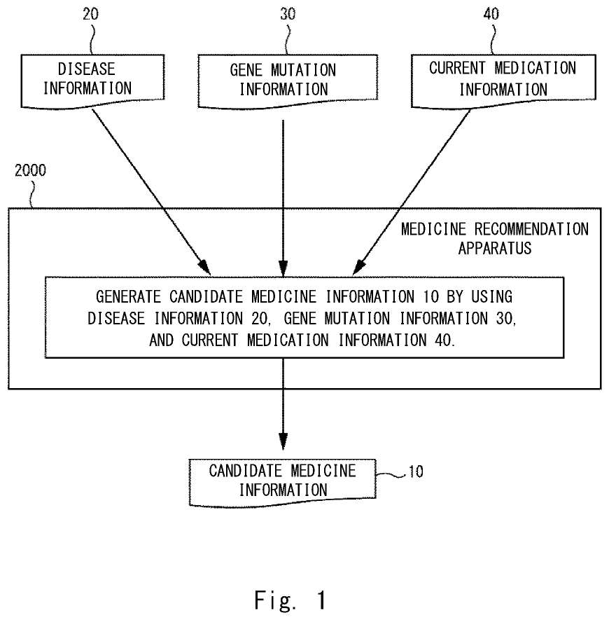

FIG. 1 shows an example of an overview of operations performed by a medicine recommendation apparatus 2000 according to a first example embodiment. Note that FIG. 1 is a diagram for facilitating the understanding of the overview of the medicine recommendation apparatus 2000, and the operations performed by the medicine recommendation apparatus 2000 are not limited to those shown in FIG. 1.

The medicine recommendation apparatus 2000 is used to generate candidate medicine information 10. The candidate medicine information 10 is information about a candidate for a medicine to be prescribed to a person or an animal or to be recommended to a person or an animal to use (hereinafter, expressed as “to be prescribed or the like”). In the following description, a person or an animal to which a medicine is prescribed or the like is referred to as a “subject”. For example, the candidate medicine information 10 indicates one or more medicines which are recommended being prescribed or the like to the subject or indicates a degree of recommendation for the prescription or like of each medicine.

For example, the candidate medicine information 10 is used as information that a doctor refers to when she/he prescribes a medicine to a person patient or an animal patient. However, situations where the candidate medicine information 10 is used are not necessarily limited to situations where a medicine is prescribed by a doctor. For example, the candidate medicine information 10 may be used by a clerk in a drugstore to select a medicine that she/he recommends to a customer.

The medicine recommendation apparatus 2000 acquires disease information 20, gene mutation information 30, and current medication information 40, and generates candidate medicine information 10 by using these acquired information items. The disease information 20 indicates, among diseases that the subject suffers from, a disease that is subject to the medicine indicated by the candidate medicine information 10. For example, in a case where a medicine for a stomach cancer is to be prescribed or the like to a subject who suffers from two diseases (the stomach cancer and a lower-back pain), the disease information 20 indicates the “stomach cancer” as the disease.

The gene mutation information 30 is information indicating a gene mutation that the subject has. Examples of gene mutations include BRAF V600E and BRAF V600K. The current medication information 40 indicates a medicine that the subject currently uses. For example, in the above-described case where the medicine for a stomach cancer is to be prescribed or the like to the subject suffering from the two diseases (the stomach cancer and the lower-back pain), it is assumed that the subject currently uses a medicine for treating the lower-back pain. In this case, the current medication information 40 indicates the medicine that the subject uses for the lower-back pain. Note that medicines that the current medication information 40 can indicate are not limited to medicines prescribed by a doctor, but may be a medicine that the subject purchases in a drugstore or the like without a prescription from a doctor (e.g., a medicine classified as a quasi-drug).

### Example of Advantageous Effect

According to the medicine recommendation apparatus 2000 in accordance with this example embodiment, the candidate medicine information 10 is generated by using the disease information 20, the gene mutation information 30, and the current medication information 40. In this way, it is possible to recommend a medicine to be prescribed or the like to a subject for a disease she/he suffers from while taking a gene mutation that the subject has and a medicine that the subject uses into consideration. Therefore, it is possible to recommend a medicine that is more suitable for the subject than the case where the gene mutation and the currently medication are not taken into consideration.

The medicine recommendation apparatus 2000 according to this example embodiment will be described hereinafter in a more detailed manner.

### Example of Functional Configuration

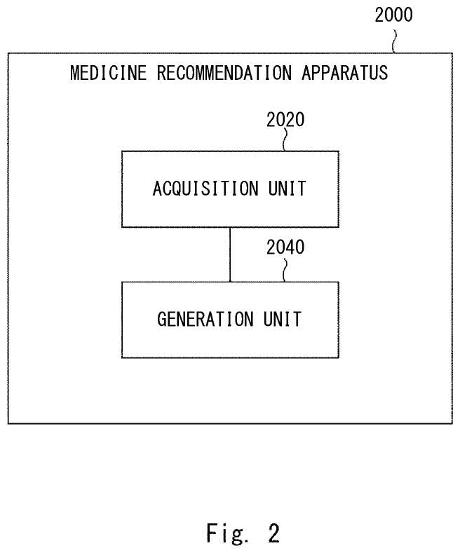

FIG. 2 is a block diagram showing an example of a functional configuration of the medicine recommendation apparatus 2000 according to the first example embodiment. The medicine recommendation apparatus 2000 includes an acquisition unit 2020 and a generation unit 2040. The acquisition unit 2020 acquires disease information 20, gene mutation information 30, and current medication information 40. The generation unit 2040 generates candidate medicine information 10 by using the disease information 20, the gene mutation information 30, and the current medication information 40.

### Example of Hardware Configuration

Each of functional components of the medicine recommendation apparatus 2000 can be implemented by hardware that implements the functional component (e.g., a hardwired electronic circuit or the like) or by a combination of hardware and software (e.g., a combination of an electronic circuit and a program for controlling it or the like). A case where each of the functional components of the medicine recommendation apparatus 2000 is implemented by a combination of hardware and software will be further described hereinafter.

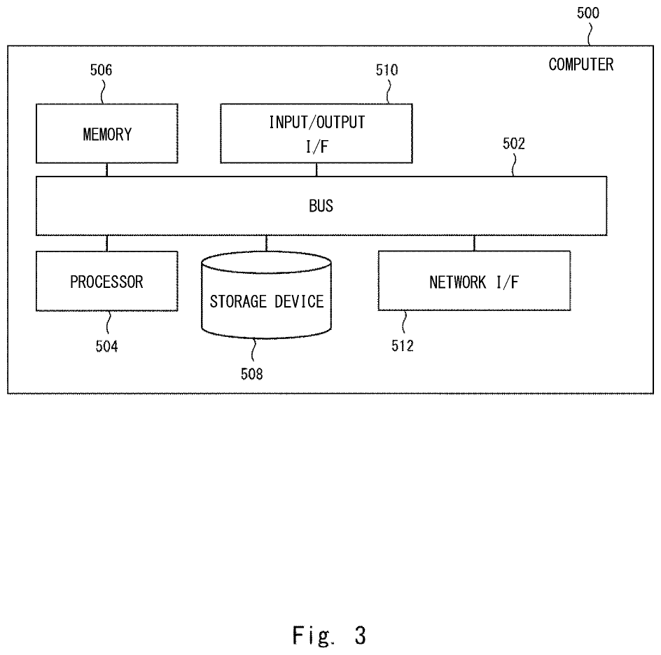

FIG. 3 is a block diagram showing an example of a hardware configuration of a computer 500 that implements the medicine recommendation apparatus 2000. The computer 500 is an arbitrary computer. For example, the computer 500 is a stationary computer such as a PC (Personal Computer) or a server machine. Alternatively, for example, the computer 500 is a mobile computer such as a smartphone or a tablet-type terminal. The computer 500 may be a special-purpose computer designed to realize the medicine recommendation apparatus 2000, or may be a general-purpose computer.

For example, each of functions of the medicine recommendation apparatus 2000 is implemented by the computer 500 by installing a predetermined application in the computer 500. The aforementioned application is composed of a program for implementing each of the function components of the medicine recommendation apparatus 2000. Note that how to acquire the aforementioned program is arbitrarily determined. For example, the program can be acquired from a storage medium (such as a DVD or a USB memory) in which the program is stored. Alternatively, the program can be acquired, for example, by downloading the program from a server apparatus that manages a storage device in which the program is stored.

The computer 500 includes a bus 502, a processor 504, a memory 506, a storage device 508, an input/output interface 510, and a network interface 512. The bus 502 is a data transmission path through which the processor 504, the memory 506, the storage device 508, the input/output interface 510, and the network interface 512 transmit and receive data to and from each other. However, the method for connecting the processor 504 and the like to each other is not limited to connections through buses.

The processor 504 is any of various types of processors such as a CPU (Central Processing Unit), a GPU (Graphics Processing Unit), or an FPGA (Field-Programmable Gate Array). The memory 506 is a primary storage device implemented by using a RAM (Random Access Memory) or the like. The storage device 508 is a secondary storage device implemented by using a hard disk drive, an SSD (Solid State Drive), a memory card, or a ROM (Read Only Memory).

The input/output interface 510 is an interface for connecting the computer 500 with an input/output device. For example, an input device such as a keyboard and an output device such as a display device are connected to the input/output interface 510.

The network interface 512 is an interface for connecting the computer 500 to a network. The network may be a LAN (Local Area Network) or a WAN (Wide Area Network).

In the storage device 508, programs for implementing respective functional components of the medicine recommendation apparatus 2000 (programs for implementing the above-described applications) are stored. The processor 504 implements each of functional components of the medicine recommendation apparatus 2000 by loading the aforementioned program onto the memory 506 and executing the loaded program.

The medicine recommendation apparatus 2000 may be implemented by one computer 500 or by a plurality of computers 500. In the latter case, the configurations of the computers 500 do not need to be identical to each other, but can be different from each other.

### Flow of Processes

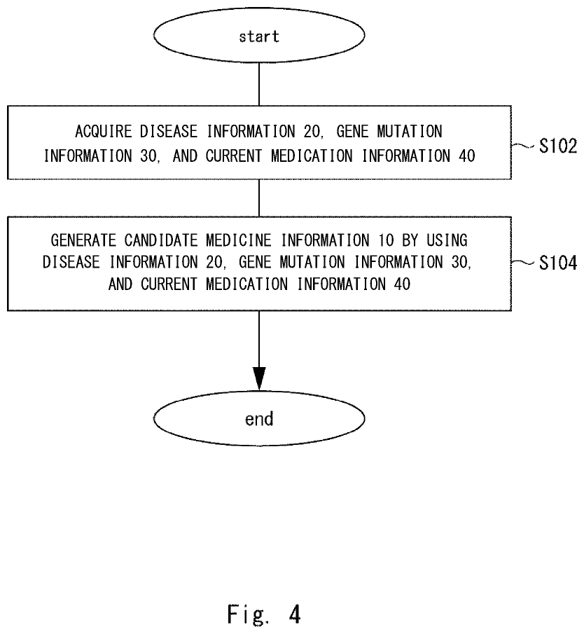

FIG. 4 is a flowchart showing an example of a flow of processes performed by the medicine recommendation apparatus 2000 according to the first example embodiment. The acquisition unit 2020 acquires disease information 20, gene mutation information 30, and current medication information 40 (S102). The generation unit 2040 generates candidate medicine information 10 by using the disease information 20, the gene mutation information 30, and the current medication information 40 (S104).

### Acquisition of Information: S102

The acquisition unit 2020 acquires disease information 20, gene mutation information 30, and current medication information 40 (S102). There are various ways by which the acquisition unit 2020 acquires these information items. For example, these information items are stored in advance in a storage device accessible from the medicine recommendation apparatus 2000. The acquisition unit 2020 acquires these information items by accessing this storage device. As a more specific example, these information items can be included in data representing a medical record of a subject (so-called an electronic medical record). In this case, the acquisition unit 2020 acquires each of the above-described information items from the electronic medical record. Note that an existing technique can be used as a technique for acquiring desired information from an electronic medical record of a specific person.

Alternatively, for example, the disease information 20, the gene mutation information 30, and the current medication information 40 may be entered by a user. Alternatively, for example, the disease information 20, the gene mutation information 30, and the current medication information 40 may be transmitted from other apparatuses to the medicine recommendation apparatus 2000.

### Generation of Candidate Medicine Information 10: S104

The generation unit 2040 generates candidate medicine information 10 by using the disease information 20, the gene mutation information 30, and the current medication information 40. For example, the disease information 20, the gene mutation information 30, and the current medication information 40 are respectively used to determine: at least one medicine that can be prescribed or the like to the subject; the presence or absence of resistance or susceptibility to the medicine, the level of resistance or susceptibility to the medicine; and a side effect(s) that may occur when the medicine is used together with current medication. Details of this feature will be described hereinafter.

### Determination of Medicine that can be Prescribed or the Like

The generation unit 2040 determines at least one medicine that can be prescribed or the like to the subject based on the disease of the subject indicated by the disease information 20. The medicine determined in this process will be referred to as a candidate medicine hereinafter.

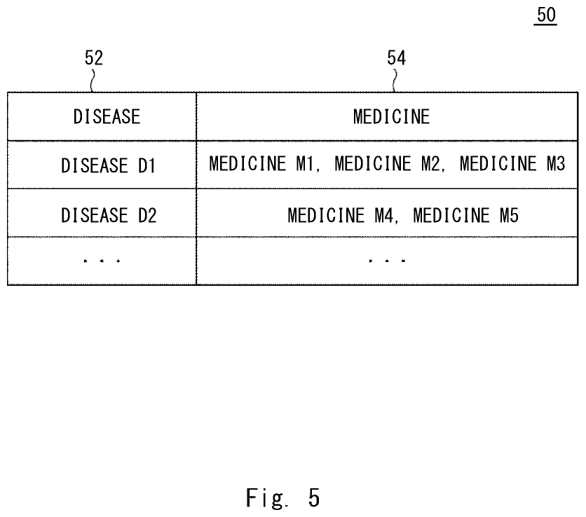

For determining a candidate medicine, for example, information that indicates diseases and medicines that can be used for the diseases in association with each other is used. This information is referred to as first knowledge information hereinafter. FIG. 5 shows an example of first knowledge information in the form of a table. The first knowledge information 50 shown in FIG. 5 has two columns named Disease 52 and Medicine 54. Disease 52 indicates identification information of a disease (a name of disease, identification code, etc.). Medicine 54 indicates identification information of medicine (a name of medicine, an identification code, etc.) that can be used for the corresponding disease 52.

For example, the generation unit 2040 determines, out of the first knowledge information 50, a record whose Disease 52 indicates the disease indicated by the disease information 20. Then, the generation unit 2040 determines a medicine indicated by Medicine 54 of the determined record as a candidate medicine.

Attributes of the subject may also be taken into consideration when a candidate medicine is determined. In this case, the acquisition unit 2020 further acquires attribute information that indicates attributes of the subject. Examples of attributes of a subject indicated by the attribute information include an age, gender, race, height, and weight of the subject, and diseases other than the disease indicated by the disease information 20. In this case, the first knowledge information 50 further indicates information about attributes.

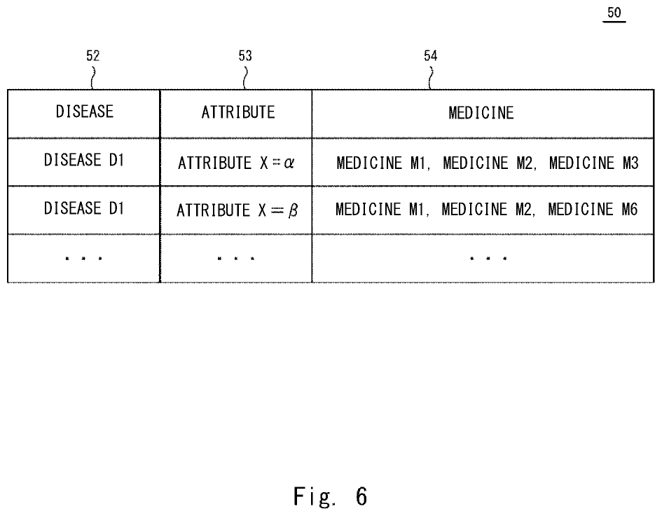

FIG. 6 shows an example of first knowledge information 50 further containing information about attributes. FIG. 6 has a column named Attribute 53. The generation unit 2040 determined, out of the first knowledge information 50, a record whose Disease 52 indicates the disease that is indicated by the disease information 20 and whose Attribute 53 indicates an attribute that matches the attribute of the subject. Then, the generation unit 2040 determines a medicine indicated by the determined record as a candidate medicine.

### Determining Resistance or Susceptibility

A person having a specific gene mutation may have resistance or susceptibility to a specific medicine due to that gene mutation. When a subject has resistance to a certain medicine, that medicine may not effective to the subject or may be less effective to the subject. On the other hand, when a subject has susceptibility to a certain medicine, that medicine may be effective to the subject or may be more effective to the subject. Therefore, the decision on whether or not a specific medicine should be prescribed or the like to a subject and the determination of the dose (i.e., an amount of prescription) of that medicine are affected by a gene mutation that the subject has.

Therefore, for each candidate medicine, based on the gene mutation of the subject indicated by the gene mutation information 30, the generation unit 2040 determines whether or not the subject has resistance or susceptibility to the candidate medicine or determine the level of resistance or susceptibility of the subject to the candidate medicine. For determining them, for example, second knowledge information that associates gene mutations, medicines, and information indicating resistance or susceptibility of persons having the gene mutations to the medicines with one another is used.

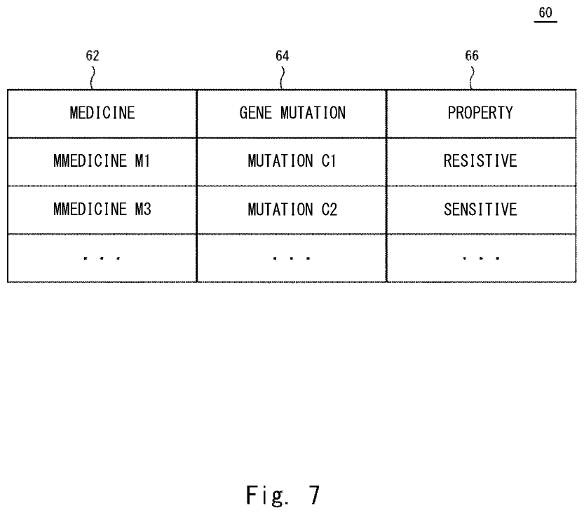

FIG. 7 shows an example of second knowledge information in the form of a table. The second knowledge information 60 shown in FIG. 7 has three columns named Gene Mutation 62, Medicine 64, and Property 66. Gene Mutation 62 indicates identification information of a gene mutation (a name of gene mutations, an identification code, etc.). Medicine 64 indicates identification information of a medicine. Property 66 indicates, for a gene mutation indicated by the corresponding gene mutation 62 and for a medicine indicated by the corresponding medicine 64, information about resistance or susceptibility of a person having the gene mutation to the medicine. For example, Property 66 indicates whether or not the person has resistance or susceptibility. Alternatively, for example, Property 66 indicates a level of resistance or susceptibility.

The level of resistance or susceptibility is represented, for example, by a recommended dose of the corresponding medicine. The dose of a medicine may be represented by an absolute amount, or may be represented by a relative amount to the recommended dose for a person who does not have the corresponding gene mutation (i.e., a relative amount to a standard dose).

For example, for each candidate medicine, the generation unit 2040 determines, out of the second knowledge information 60, a record whose Gene Mutation 62 indicates the gene mutation that is indicated by the gene mutation information 30 and whose Medicine 64 indicates that candidate medicine. Then, the generation unit 2040 determines, by referring to the property 66 indicated by the determined, whether or not the subject has resistance or susceptibility to that candidate medicine, or determine the level of resistance or susceptibility.

Attributes of the subject may also be taken into consideration when resistance or susceptibility is determined. In this case, the acquisition unit 2020 also acquires the above-described attribute information. Further, in this case, the second knowledge information 60 further indicates information about attributes.

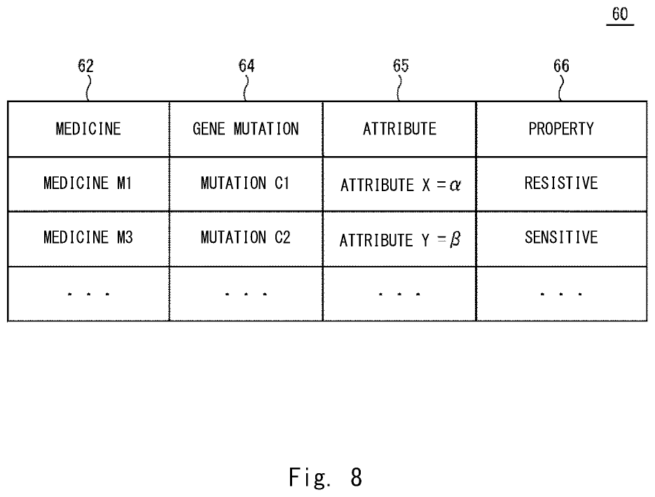

FIG. 8 shows an example of second knowledge information 60 further including information about attributes. The second knowledge information 60 in FIG. 8 has a column named Attribute 65. The generation unit 2040 determined, for each candidate medicine, out of the second knowledge information 60, a record whose Gene Mutation 62 indicates the gene mutation indicated by the gene mutation information 30, whose Medicine 64 indicates that candidate medicine, and whose Attribute 65 indicates an attribute that matches an attribute of the subject. Then, the generation unit 2040 determines, by referring to the column

Property 66 in the determine record, whether or not the subject has resistance or susceptibility to that candidate medicine, or determines the level of resistance or susceptibility.

### Determining Side Effect

The generation unit 2040 determines, for each candidate medicine, a side effect that can occur when the candidate medicine is used together with a medicine indicated by the current medication information 40 (i.e., a medicine that the subject is using). For determining a side effect, for example, information indicating two medicines that are used together and a side effect that can occur when these medicines are used together in association with each other is used. This information is referred to as third knowledge information hereinafter.

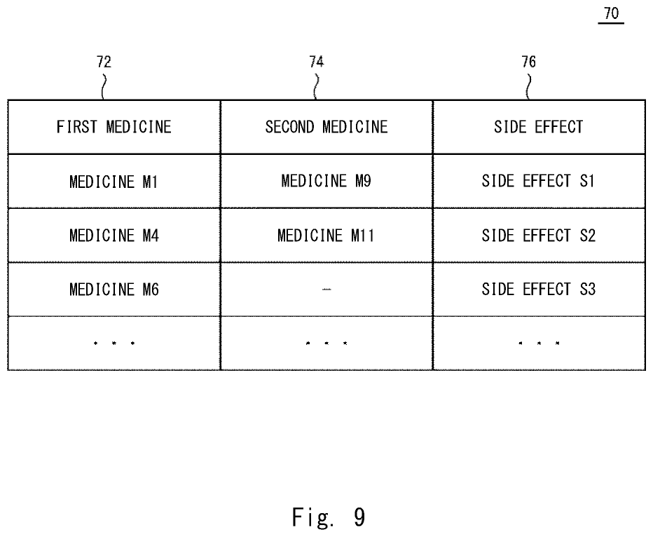

FIG. 9 shows an example of third knowledge information in the form of a table. The third knowledge information 70 shown in FIG. 9 has three columns named First Medicine 72, Second medicine 74, and Side Effect 76. First Medicine 72 indicates identification information of candidate medicines. Second Medicine 74 indicates identification information of the current medication. Side Effect 76 indicates a side effect that can occur when two medicines indicated by the corresponding First Medicine 72 and Second Medicine 74 are used together.

For example, for each candidate medicine, the generation unit 2040 determines, out of the third knowledge information 70, a record whose First Medicine indicates that candidate medicine and whose Second Medicine 74 indicates a medicine that is indicated by the current medication information 40. Then, the generation unit 2040 determines, by referring to Side Effect 76 in the determined record, a side effect that can occur when the candidate medicine and the medicine indicated by the current medication information 40 are used together.

Note that the third knowledge information 70 may also indicate side effects of candidate medicines themselves. A record indicating a side effect of a candidate medicine itself can be realized as one whose Second Medicine 74 is empty.

Attributes of the subject may also be taken into consideration when a side effect is determined. In this case, the acquisition unit 2020 also acquires the above-described attribute information. Further, in this case, the third knowledge information 70 further indicates information about attributes.

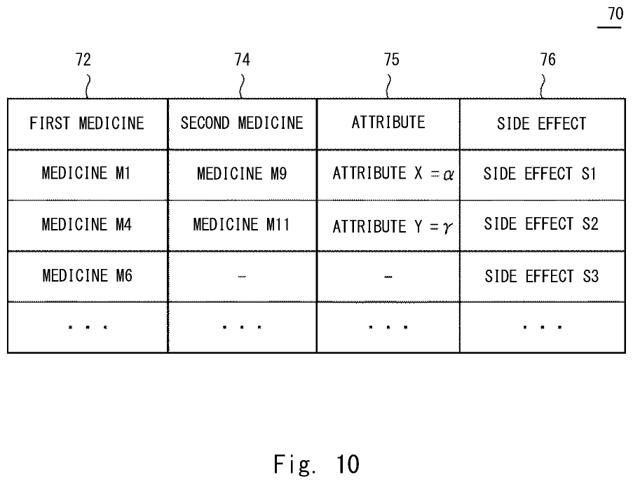

FIG. 10 shows an example of third knowledge information 70 further including information about attributes. The third knowledge information 70 in FIG. 10 has a column named Attribute 75. For each candidate medicine, the generation unit 2040 determines, out of the third knowledge information 70, a record whose First Medicine 72 indicates that candidate medicine, whose Second Medicine 74 indicates a medicine that is indicated by the current medication information 40, and whose Attribute 75 indicates an attribute that matches an attribute of the subject. Then, the generation unit 2040 determines, by referring to Side Effect 76 in the determined record, a side effect that can occur when the candidate medicine and the medicine indicated by the current medication information 40 are used together.

### Generation of Candidate Medicine Information 10

The generation unit 2040 generates candidate medicine information 10 based on the results of the above-described determination processes. The candidate medicine information 10 indicates at least one of the above-described candidate medicines as a medicine that is recommended to be prescribed or the like. For example, the generation unit 2040 computes, for each candidate medicine, a degree of recommendation of the candidate medicine (hereinafter, also called a recommendation score) based on the resistance or susceptibility of the subject to the candidate medicine and the side effect that can occur when the candidate medicine and another medicine are used together. The generation unit 2040 generates candidate medicine information 10 based on the recommendation score computed for each candidate medicine. A method for computing a recommendation score will be described later.

For example, the candidate medicine information 10 is generated so that it indicates only candidate medicines whose recommendation scores are equal to or greater than a predetermined threshold. In this case, the generation unit 2040 determines, by comparing the recommendation score of each candidate medicine with a threshold, candidate medicines whose recommendation scores are equal to or greater than the threshold. Then, the generation unit 2040 generates candidate medicine information 10 that indicates candidate medicines each of which is determined to have a recommendation score equal to or greater than the threshold. Note that the candidate medicine information 10 may indicate candidate medicines whose recommendation scores are equal to or greater than the threshold together with their recommendation scores.

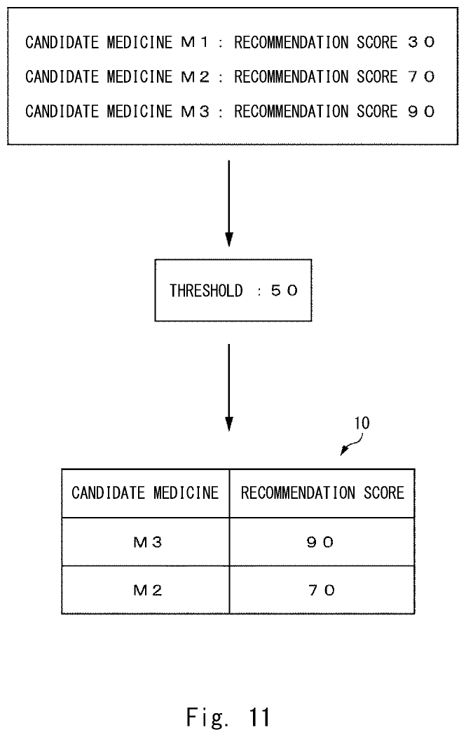

FIG. 11 conceptually shows a process for generating candidate medicine information 10. In the example shown by FIG. 11, the candidate medicines are candidate medicine M1, M2 and M3. The recommendation scores of the candidate medicine M1, M2 and M3 are 30, 70 and 90, respectively. Further, the threshold of the recommendation score to determine candidate medicines to be included in the candidate medicine information 10 is 50. That is, only candidate medicines whose recommendation scores are 50 or greater are included in the candidate medicine information 10.

In this example, the medicines M2 and M3 have a recommendation score of 50 or greater. Therefore, the candidate medicine information 10 indicates the candidate medicines M2 and M3, and their recommendation scores. Note that it is preferable that candidate medicines indicated by the candidate medicine information 10 are sorted according to the values of the recommendation scores.

The candidate medicine information 10 may indicate ranks that are determined based on the values of the recommendation scores together with or instead of the recommendation scores. In this case, the range of the recommendation score is divided into a plurality of numerical ranges that do not overlap each other, and a rank is assigned to each of the divided numerical ranges. The generation unit 2040 determines, for each of candidate medicines to be included in the candidate medicine information 10, a numerical range to which its recommendation score belongs. Then, the generation unit 2040 generates candidate medicine information 10 so that each of candidate medicines is indicated together with a rank determined for that candidate medicine.

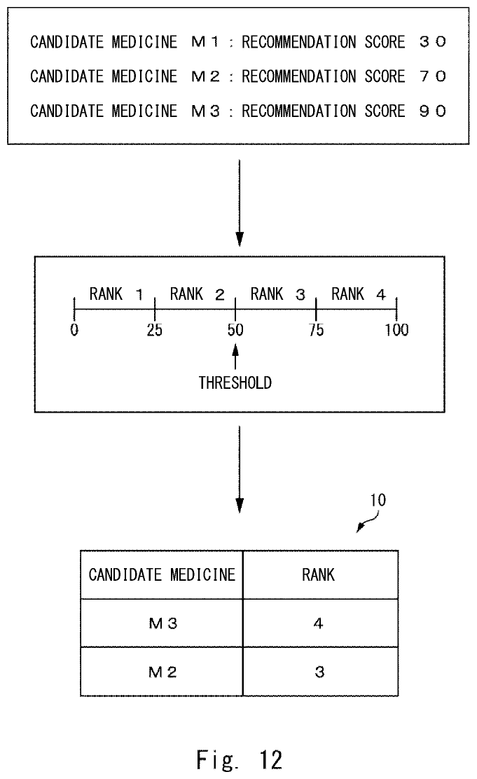

FIG. 12 shows an example of a case where candidate medicine information 10 indicates ranks. In this example, the range of the recommendation score is between 0 (inclusive) and 100 (inclusive). Further, ranks 1, 2, 3, and 4 are assigned to a range from 0 (inclusive) to 25 (not inclusive), a range from 25 (inclusive) to 50 (not inclusive), a range from 50 (inclusive) to 75 (not inclusive), and a range from 75 (inclusive) to 100 (inclusive), respectively. Other assumptions are the same as those in the example shown in FIG. 11.

As described above, the candidate medicines included in the candidate medicine information 10 are the candidate medicines M2 and M3. Since the recommendation score of the candidate medicine M2 is 70, its rank is Rank 3. Since the recommendation score of the candidate medicine M3 is 90, its rank is Rank 4. Therefore, the candidate medicine information 10 in FIG. 12 also indicates Ranks 3 and 4 for the candidate medicines M2 and M3, respectively.

The candidate medicine information 10 may indicate all the candidate medicines, instead of indicating only the candidate medicines each of which has a recommendation score equal to or greater than the threshold. In this case, for example, the generation unit 2040 generates candidate medicine information 10 indicating all the candidate medicines together with their recommendation scores. Note that, in this case, as described above, ranks, which are determined based on the values of recommendation scores, may be indicated together with or instead of the recommendation scores.

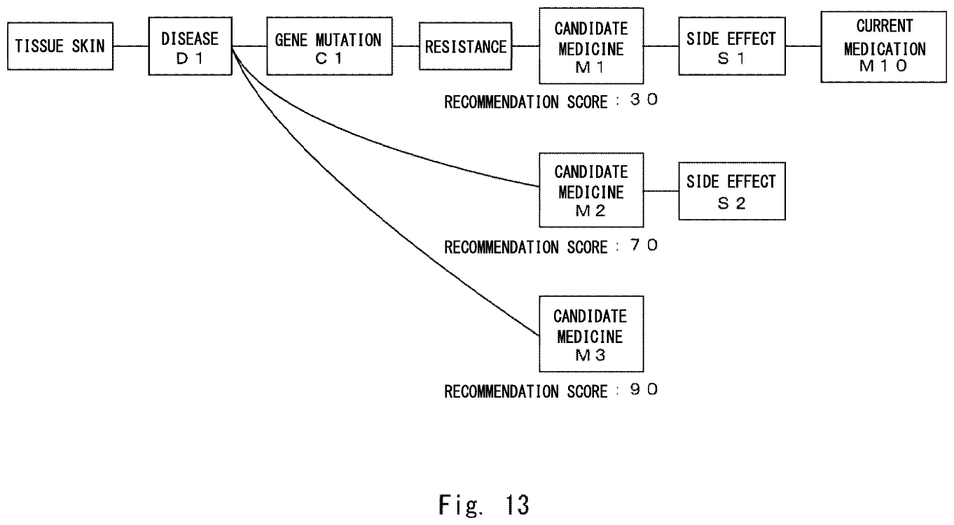

Further, the candidate medicine information 10 may also include, in addition to the information about candidate medicines, various information items that are used to generate candidate medicines or their recommended ranks. For example, the candidate medicine information 10 may include information that associates a gene mutation that the subject has with resistance or susceptibility of the subject to the candidate medicine, or information that associates a medicine that the subject is using (the current medication of the subject) with a side effect that can occur when this medicine is used together with the candidate medicine. FIG. 13 shows an example of candidate medicine information 10 including information about resistance or the like and side effects. In this example, a disease that the subject suffers from is represented by D1. Further, a gene mutation that the subject has is represented by C1. Further, candidate medicines which can be prescribed to the subject for the disease D1 are candidate medicines M1, M2 and M3.

A mark “Resistance” is shown between Candidate Medicine M1 and Gene Mutation C1. This indicates that the subject has resistance to the candidate medicine M1 due to the gene mutation C1. That is, the candidate medicine M1 is a less effective for the subject.

Further, FIG. 13 shows a medicine M10 as a medicine that the subject is already using. Further, Side Effect S1 is shown between Medicine M1 and Medicine M10. This means that the side effect S1 occurs when the medicines M1 and M10 are used together.

Further, Side Effect S2 is shown for Candidate Medicine M2 without being associated with any other medicine. This means that the side effect S2 occurs by the candidate medicine M2 itself.

When displaying on a display device a screen that represents the candidate medicine information 10 shown by FIG. 13, it is preferable to allow, for each piece of information shown by FIG. 13, to access more detailed information about that piece of information. Therefore, it is preferable include links to detailed information in the screen. For example, the screen may be configured so that when a mark “Resistance” is selected, detailed information about the resistance is provided (e.g., detailed information is displayed in a pop-up manner). The same applies to information pieces other than “Resistance”.

By viewing the information shown by FIG. 13, a doctor can further understand not only a degree of recommendation for each candidate medicine but also the ground for the degree of recommendation of the candidate medicine.

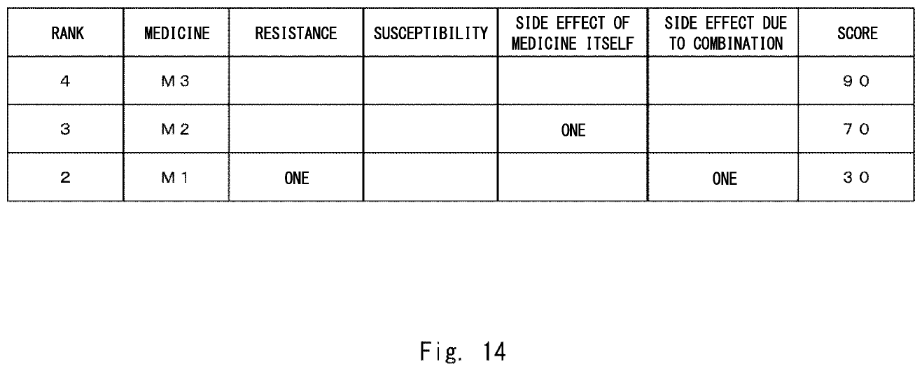

FIG. 14 is a second diagram showing an example of candidate medicine information 10 including information about resistance and side effects. The assumptions are the same as those in the example shown in FIG. 13.

In the example shown by FIG. 14, candidate medicine information 10 is shown in the form of a table. Specifically, for each candidate medicine, the number of pieces of information about resistance or susceptibility obtained from the gene mutation information 30 and the number of pieces of information about side effects obtained from the current medication information 40 for the candidate medicine are shown. Note that the number of pieces of information is represented, for example, by the number of corresponding records in the gene mutation information 30 or the current medication information 40. Further, the table shown by FIG. 14 shows a score and a rank for each candidate medicine.

When displaying on a display device a screen that represents the candidate medicine information 10 shown by FIG. 14, it is also preferable to allow, for each piece of information shown by FIG. 14, to access more detailed information about that piece of information. Therefore, it is preferable to include links to detailed information in the screen. For example, the image may be configured so that when the number of pieces included in the table, such as “One”, is selected, detailed information is provided (e.g., detailed information is displayed in a pop-up manner).

### Computation of Recommendation Score

The generation unit 2040 computes a recommendation score for each candidate medicine and generates candidate medicine information 10 based on the computed recommendation scores. It is preferable to reduce the recommendation score of a candidate medicine when the subject has resistance to that candidate medicine. That is, regarding a candidate medicine to which the subject has resistance, the generation unit 2040 makes the recommendation score smaller than that in the case in which the subject does not have resistance to that candidate medicine. Further, the higher resistance the subject has to a candidate medicine, the smaller the recommendation score for that candidate medicine is made.

It is preferable to increase the recommendation score of a candidate medicine when the subject has susceptibility to that candidate medicine. That is, regarding a candidate medicine to which the subject has susceptibility, the generation unit 2040 makes the recommendation score greater than that in the case in which the subject does not have susceptibility to that candidate medicine. Further, the higher susceptibility the subject has to a candidate medicine, the greater the recommendation score for that candidate medicine is made.

It is preferable to reduce the recommendation score of a candidate medicine that causes a side effect when the subject uses it. That is, regarding a candidate medicine that is determined to be possible to cause a side effect when being used by the subject, the generation unit 2040 makes the recommendation score smaller than that in the case in which it is determined not to cause a side effect when being used by the subject. For example, the larger the number of side effects obtained from the third knowledge information 70 is, the smaller the recommendation score is made. Further, for each type of side effect, the level of an effect (e.g., the seriousness of a side effect) may be defined in advance. In this case, the generation unit 2040 computes a recommendation score according to the level of the effect.

Based on the above-described matters, a recommendation score is computed by using, for example, the following Equation 1.

$$\begin{matrix}[ Equation 1 ] & \\ S t [ i ] = α * S 1 [ i ] + β * S 2 [ i ] + γ * S3 [ i ] & ( 1 )\end{matrix}$$

In Equation (1), St[i] represents a recommendation score of a candidate medicine having an identifier i (a candidate medicine i). S1[i] represents a score computed based on the resistance of the subject to the candidate medicine i. S2[i] represents a score computed based on the susceptibility of the subject to candidate medicine i. S3[i] represents a score computed based on the side effect that can occur when the subject uses the candidate medicine i. Further, α, β, and γ are weights assigned to respective scores. That is, in Equation (1), a recommendation score is computed as a weighted sum of the score computed based on the resistance, the score computed based on the susceptibility, and the score computed based on the side effect.

### Output by Medicine Recommendation Apparatus 2000

The medicine recommendation apparatus 2000 outputs candidate medicine information 10 by using an arbitrary method. For example, the medicine recommendation apparatus 2000 puts candidate medicine information 10 into an arbitrary storage device accessible from the medicine recommendation apparatus 2000. Alternatively, for example, the medicine recommendation apparatus 2000 causes a display device accessible from the medicine recommendation apparatus 2000 to display candidate medicine information 10. Alternatively, for example, the medicine recommendation apparatus 2000 may transmit candidate medicine information 10 to an arbitrary apparatus accessible from the medicine recommendation apparatus 2000.

Although the present invention is described above with reference to example embodiments, the present invention is not limited to the above-described example embodiments. Various modifications that can be understood by those skilled in the art can be made to the configuration and details of the present invention within the scope of the invention.

Note that, in the above-described examples, the program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM, CD-R, CD-R/W, and semiconductor memories (such as mask ROM, PROM (Programmable ROM), EPROM (Erasable PROM), flash ROM, RAM, etc.). Further, the program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

The whole or part of the example embodiments disclosed above can be described as, but not limited to, the following supplementary notes.

### Supplementary Note 1

A medicine recommendation apparatus comprising:
an acquisition unit configured to acquire disease information that is regarding a disease that a subject suffers from, gene mutation information that is regarding a gene mutation of the subject, and current medication information that is regarding a medicine that the subject is using; and
a generation unit configured to generate candidate medicine information by using the disease information, the gene mutation information, and the current medication information, the candidate medicine information being information regarding a candidate for a medicine to be recommended to the subject to use or to be prescribed to the subject.

### Supplementary Note 2

The medicine recommendation apparatus according to supplementary note 1,
wherein the generation unit performs:
determining one or more candidate medicines use of which can be recommended to the subject or which can be prescribed to the subject by using first knowledge information and the disease information, the first knowledge information indicating a disease and a medicine that can be used for the disease in association with each other;
determining resistance or susceptibility of the subject to each of the candidate medicines by using second knowledge information and the gene mutation information, the second knowledge information indicating information regarding, for each combination of a gene mutation and a medicine, resistance or susceptibility of a person having the gene mutation to the medicine in association with each other;
determining, for each of the candidate medicines, a side effect that can occur in the subject when the medicine is used by using third knowledge information and the current medication information, the third knowledge information being information regarding a side effect that can occur when a plurality of medicines are used together; and
generating the candidate medicine information indicating the one or more candidate medicines based on resistance or susceptibility of the subject to each of the candidate medicines and side effects each of which can occur in the subject when a respective one of the candidate medicines is used.

### Supplementary Note 3

The medicine recommendation apparatus according to supplementary note 2,
wherein the generation unit performs:
computing, for each of the candidate medicines, a recommendation score based on resistance or susceptibility of the subject to the candidate medicine and a side effect that can occur in the subject when the candidate medicine is used, the recommendation score representing a degree of recommendation of the candidate medicine; and
generating the candidate medicine information indicating the candidate medicine for which the recommendation score is equal to or greater than a threshold or the candidate medicine information indicating each of the candidate medicines together with the recommendation score thereof.

### Supplementary Note 4

The medicine recommendation apparatus according to supplementary note 2 or 3,
wherein the acquisition unit acquires attribute information indicating an attribute of the subject,
wherein the second knowledge information indicates, for each combination of a gene mutation, a medicine, and an attribute, information regarding the gene mutation and resistance or susceptibility of a person having the attribute to the medicine in association with each other, and
wherein the generation unit determines, for each of the candidate medicines, resistance or susceptibility of the subject to the candidate medicine by using the gene mutation information and the attribute information.

### Supplementary Note 5

The medicine recommendation apparatus according to any one of supplementary notes 2 to 4,
wherein the acquisition unit acquires attribute information indicating an attribute of the subject,
wherein the third knowledge information indicates, for each combination of a plurality of medicines and an attribute, a side effect that can occur when a person having the attribute uses the plurality of medicines together, and
wherein the generation unit determines, for each of the candidate medicines, a side effect that can occur in the subject when the candidate medicine is used by using the gene mutation information and the attribute information.

### Supplementary Note 6

A control method performed by a computer, comprising:
an acquisition step of acquiring disease information that is regarding a disease that a subject suffers from, gene mutation information that is regarding a gene mutation of the subject, and current medication information that is regarding a medicine that the subject is using; and
a generation step of generating candidate medicine information by using the disease information, the gene mutation information, and the current medication information, the candidate medicine information being information regarding a candidate for a medicine to be recommended to the subject to use or to be prescribed to the subject.

### Supplementary Note 7

The control method according to supplementary note 6,
wherein in the generation step:
determining one or more candidate medicines use of which can be recommended to the subject or which can be prescribed to the subject by using first knowledge information and the disease information, the first knowledge information indicating a disease and a medicine that can be used for the disease in association with each other;
determining resistance or susceptibility of the subject to each of the candidate medicines by using second knowledge information and the gene mutation information, the second knowledge information indicating information regarding, for each combination of a gene mutation and a medicine, resistance or susceptibility of a person having the gene mutation to the medicine in association with each other;
determining, for each of the candidate medicines, a side effect that can occur in the subject when the medicine is used by using third knowledge information and the current medication information, the third knowledge information being information regarding a side effect that can occur when a plurality of medicines are used together; and
generating the candidate medicine information indicating the one or more candidate medicines based on resistance or susceptibility of the subject to each of the candidate medicines and side effects each of which can occur in the subject when a respective one of the candidate medicines is used.

### Supplementary Note 8

The control method according to supplementary note 7,
wherein in the generation step:
computing, for each of the candidate medicines, a recommendation score based on resistance or susceptibility of the subject to the candidate medicine and a side effect that can occur in the subject when the candidate medicine is used, the recommendation score representing a degree of recommendation of the candidate medicine; and
generating the candidate medicine information indicating the candidate medicine for which the recommendation score is equal to or greater than a threshold or the candidate medicine information indicating each of the candidate medicines together with the recommendation score thereof.

### Supplementary Note 9

The control method according to supplementary note 7 or 8,
wherein in the acquisition step, acquiring attribute information indicating an attribute of the subject,
wherein the second knowledge information indicates, for each combination of a gene mutation, a medicine, and an attribute, information regarding the gene mutation and resistance or susceptibility of a person having the attribute to the medicine in association with each other, and
wherein in the generation step, determining, for each of the candidate medicines, resistance or susceptibility of the subject to the candidate medicine by using the gene mutation information and the attribute information.

### Supplementary Note 10

The control method according to any one of supplementary notes 7 to 9,
wherein in the acquisition step, acquiring attribute information indicating an attribute of the subject,
wherein the third knowledge information indicates, for each combination of a plurality of medicines and an attribute, a side effect that can occur when a person having the attribute uses the plurality of medicines together, and
wherein in the generation step, determining, for each of the candidate medicines, a side effect that can occur in the subject when the candidate medicine is used by using the gene mutation information and the attribute information.

### Supplementary Note 11

A computer readable medium storing a program that causes a computer to execute:
an acquisition step of acquiring disease information that is regarding a disease that a subject suffers from, gene mutation information that is regarding a gene mutation of the subject, and current medication information that is regarding a medicine that the subject is using; and
a generation step of generating candidate medicine information by using the disease information, the gene mutation information, and the current medication information, the candidate medicine information being information regarding a candidate for a medicine to be recommended to the subject to use or to be prescribed to the subject.

### Supplementary Note 12

The computer readable medium according to supplementary note 11,
wherein in the generation step:
determining one or more candidate medicines use of which can be recommended to the subject or which can be prescribed to the subject by using first knowledge information and the disease information, the first knowledge information indicating a disease and a medicine that can be used for the disease in association with each other;
determining resistance or susceptibility of the subject to each of the candidate medicines by using second knowledge information and the gene mutation information, the second knowledge information indicating information regarding, for each combination of a gene mutation and a medicine, resistance or susceptibility of a person having the gene mutation to the medicine in association with each other;
determining, for each of the candidate medicines, a side effect that can occur in the subject when the medicine is used by using third knowledge information and the current medication information, the third knowledge information being information regarding a side effect that can occur when a plurality of medicines are used together; and
generating the candidate medicine information indicating the one or more candidate medicines based on resistance or susceptibility of the subject to each of the candidate medicines and side effects each of which can occur in the subject when a respective one of the candidate medicines is used.

### Supplementary Note 13

The computer readable medium according to supplementary note 12,
wherein in the generation step:
computing, for each of the candidate medicines, a recommendation score based on resistance or susceptibility of the subject to the candidate medicine and a side effect that can occur in the subject when the candidate medicine is used, the recommendation score representing a degree of recommendation of the candidate medicine; and
generating the candidate medicine information indicating the candidate medicine for which the recommendation score is equal to or greater than a threshold or the candidate medicine information indicating each of the candidate medicines together with the recommendation score thereof.

### Supplementary Note 14

The computer readable medium according to supplementary note 12 or 13,
wherein in the acquisition step, acquiring attribute information indicating an attribute of the subject,
wherein the second knowledge information indicates, for each combination of a gene mutation, a medicine, and an attribute, information regarding the gene mutation and resistance or susceptibility of a person having the attribute to the medicine in association with each other, and
wherein in the generation step, determining, for each of the candidate medicines, resistance or susceptibility of the subject to the candidate medicine by using the gene mutation information and the attribute information.

### Supplementary Note 15

The computer readable medium according to any one of supplementary notes 12 to 14,
wherein in the acquisition step, acquiring attribute information indicating an attribute of the subject,
wherein the third knowledge information indicates, for each combination of a plurality of medicines and an attribute, a side effect that can occur when a person having the attribute uses the plurality of medicines together, and
wherein in the generation step, determining, for each of the candidate medicines, a side effect that can occur in the subject when the candidate medicine is used by using the gene mutation information and the attribute information.

### REFERENCE SIGNS LIST

10 CANDIDATE MEDICINE INFORMATION
20 DISEASE INFORMATION
30 GENE MUTATION INFORMATION
40 CURRENT MEDICATION INFORMATION
50 FIRST KNOWLEDGE INFORMATION
52 DISEASE
53 ATTRIBUTE
54 MEDICINE
60 SECOND KNOWLEDGE INFORMATION
62 GENE MUTATION
64 MEDICINE
65 ATTRIBUTE
66 PROPERTY
70 THIRD KNOWLEDGE INFORMATION
72 FIRST MEDICINE
74 SECOND MEDICINE
75 ATTRIBUTE
76 SIDE EFFECT
500 COMPUTER
502 BUS
504 PROCESSOR
506 MEMORY
508 STORAGE DEVICE
510 INPUT/OUTPUT INTERFACE
512 NETWORK INTERFACE
2000 MEDICINE RECOMMENDATION APPARATUS
2020 ACQUISITION UNIT
2040 GENERATION UNIT

1. A medicine recommendation apparatus comprising:
at least one memory that is configured to store instructions; and
at least one processor that is configured to execute the instructions to:
acquire disease information that is regarding a disease that a subject suffers from, gene mutation information that is regarding a gene mutation of the subject, and current medication information that is regarding a medicine that the subject is using; and
generate candidate medicine information by using the disease information, the gene mutation information, and the current medication information, the candidate medicine information being information regarding a candidate for a medicine to be recommended to the subject to use or to be prescribed to the subject.

2. The medicine recommendation apparatus according to claim 1,
wherein the generation of the candidate medicine information further includes:
determining one or more candidate medicines use of which can be recommended to the subject or which can be prescribed to the subject by using first knowledge information and the disease information, the first knowledge information indicating a disease and a medicine that can be used for the disease in association with each other;
determining resistance or susceptibility of the subject to each of the candidate medicines by using second knowledge information and the gene mutation information, the second knowledge information indicating information regarding, for each combination of a gene mutation and a medicine, resistance or susceptibility of a person having the gene mutation to the medicine in association with each other;
determining, for each of the candidate medicines, a side effect that can occur in the subject when the medicine is used by using third knowledge information and the current medication information, the third knowledge information being information regarding a side effect that can occur when a plurality of medicines are used together; and
generating the candidate medicine information indicating the one or more candidate medicines based on resistance or susceptibility of the subject to each of the candidate medicines and side effects each of which can occur in the subject when a respective one of the candidate medicines is used.

3. The medicine recommendation apparatus according to claim 2,
wherein the generation of the candidate information further includes:
computing, for each of the candidate medicines, a recommendation score based on resistance or susceptibility of the subject to the candidate medicine and a side effect that can occur in the subject when the candidate medicine is used, the recommendation score representing a degree of recommendation of the candidate medicine; and
generating the candidate medicine information indicating the candidate medicine for which the recommendation score is equal to or greater than a threshold or the candidate medicine information indicating each of the candidate medicines together with the recommendation score thereof.

4. The medicine recommendation apparatus according to claim 2,
wherein the at least one processor is configured to execute the instructions further to acquire attribute information indicating an attribute of the subject,
wherein the second knowledge information indicates, for each combination of a gene mutation, a medicine, and an attribute, information regarding the gene mutation and resistance or susceptibility of a person having the attribute to the medicine in association with each other, and
wherein the generation of the candidate medicine information further includes determining, for each of the candidate medicines, resistance or susceptibility of the subject to the candidate medicine by using the gene mutation information and the attribute information.

5. The medicine recommendation apparatus according to claim 2,
wherein the at least one processor is configured to execute the instructions further to acquire attribute information indicating an attribute of the subject,
wherein the third knowledge information indicates, for each combination of a plurality of medicines and an attribute, a side effect that can occur when a person having the attribute uses the plurality of medicines together, and
wherein the generation of the candidate medicine information further includes determining, for each of the candidate medicines, a side effect that can occur in the subject when the candidate medicine is used by using the gene mutation information and the attribute information.

6. A control method performed by a computer, comprising:
acquiring disease information that is regarding a disease that a subject suffers from, gene mutation information that is regarding a gene mutation of the subject, and current medication information that is regarding a medicine that the subject is using; and
generating candidate medicine information by using the disease information, the gene mutation information, and the current medication information, the candidate medicine information being information regarding a candidate for a medicine to be recommended to the subject to use or to be prescribed to the subject.

7. The control method according to claim 6,
wherein the generation of the candidate information further includes:
determining one or more candidate medicines use of which can be recommended to the subject or which can be prescribed to the subject by using first knowledge information and the disease information, the first knowledge information indicating a disease and a medicine that can be used for the disease in association with each other;
determining resistance or susceptibility of the subject to each of the candidate medicines by using second knowledge information and the gene mutation information, the second knowledge information indicating information regarding, for each combination of a gene mutation and a medicine, resistance or susceptibility of a person having the gene mutation to the medicine in association with each other;
determining, for each of the candidate medicines, a side effect that can occur in the subject when the medicine is used by using third knowledge information and the current medication information, the third knowledge information being information regarding a side effect that can occur when a plurality of medicines are used together; and
generating the candidate medicine information indicating the one or more candidate medicines based on resistance or susceptibility of the subject to each of the candidate medicines and side effects each of which can occur in the subject when a respective one of the candidate medicines is used.

8. The control method according to claim 7,
wherein the generation of the candidate information further includes:
computing, for each of the candidate medicines, a recommendation score based on resistance or susceptibility of the subject to the candidate medicine and a side effect that can occur in the subject when the candidate medicine is used, the recommendation score representing a degree of recommendation of the candidate medicine; and
generating the candidate medicine information indicating the candidate medicine for which the recommendation score is equal to or greater than a threshold or the candidate medicine information indicating each of the candidate medicines together with the recommendation score thereof.

9. The control method according to claim 7, further comprising:
acquiring attribute information indicating an attribute of the subject,
wherein the second knowledge information indicates, for each combination of a gene mutation, a medicine, and an attribute, information regarding the gene mutation and resistance or susceptibility of a person having the attribute to the medicine in association with each other, and
wherein the generation of the candidate medicine information further includes determining, for each of the candidate medicines, resistance or susceptibility of the subject to the candidate medicine by using the gene mutation information and the attribute information.

10. The control method according to claim 7, comprising:
acquiring attribute information indicating an attribute of the subject,
wherein the third knowledge information indicates, for each combination of a plurality of medicines and an attribute, a side effect that can occur when a person having the attribute uses the plurality of medicines together, and
wherein the generation of the candidate medicine information includes determining, for each of the candidate medicines, a side effect that can occur in the subject when the candidate medicine is used by using the gene mutation information and the attribute information.

11. A computer readable medium storing a program that causes a computer to execute:
acquiring disease information that is regarding a disease that a subject suffers from, gene mutation information that is regarding a gene mutation of the subject, and current medication information that is regarding a medicine that the subject is using; and
generating candidate medicine information by using the disease information, the gene mutation information, and the current medication information, the candidate medicine information being information regarding a candidate for a medicine to be recommended to the subject to use or to be prescribed to the subject.

12. The computer readable medium according to claim 11,
wherein the generation of the candidate medicine information futher includes:
determining one or more candidate medicines use of which can be recommended to the subject or which can be prescribed to the subject by using first knowledge information and the disease information, the first knowledge information indicating a disease and a medicine that can be used for the disease in association with each other;
determining resistance or susceptibility of the subject to each of the candidate medicines by using second knowledge information and the gene mutation information, the second knowledge information indicating information regarding, for each combination of a gene mutation and a medicine, resistance or susceptibility of a person having the gene mutation to the medicine in association with each other;
determining, for each of the candidate medicines, a side effect that can occur in the subject when the medicine is used by using third knowledge information and the current medication information, the third knowledge information being information regarding a side effect that can occur when a plurality of medicines are used together; and
generating the candidate medicine information indicating the one or more candidate medicines based on resistance or susceptibility of the subject to each of the candidate medicines and side effects each of which can occur in the subject when a respective one of the candidate medicines is used.

13. The computer readable medium according to claim 12,
wherein the generation of the candidate medicine information includes:
computing, for each of the candidate medicines, a recommendation score based on resistance or susceptibility of the subject to the candidate medicine and a side effect that can occur in the subject when the candidate medicine is used, the recommendation score representing a degree of recommendation of the candidate medicine; and
generating the candidate medicine information indicating the candidate medicine for which the recommendation score is equal to or greater than a threshold or the candidate medicine information indicating each of the candidate medicines together with the recommendation score thereof.

14. The computer readable medium according to claim 12, further comprising:
acquiring attribute information indicating an attribute of the subject,
wherein the second knowledge information indicates, for each combination of a gene mutation, a medicine, and an attribute, information regarding the gene mutation and resistance or susceptibility of a person having the attribute to the medicine in association with each other, and
wherein the generation of the candidate medicine information includes determining, for each of the candidate medicines, resistance or susceptibility of the subject to the candidate medicine by using the gene mutation information and the attribute information.

15. The computer readable medium according to claim 12, further comprising:
acquiring attribute information indicating an attribute of the subject,
wherein the third knowledge information indicates, for each combination of a plurality of medicines and an attribute, a side effect that can occur when a person having the attribute uses the plurality of medicines together, and
wherein the generation of the candidate medicine information includes determining, for each of the candidate medicines, a side effect that can occur in the subject when the candidate medicine is used by using the gene mutation information and the attribute information.
